(19) United States
(12) Patent Application Publication
(10) Pub. No.: US 2017/0128332 A1
(43) Pub. Date: May 11, 2017

(54) ACTIVATION AND STABILIZATION OF BASIC ALUMINUM CHLORIDE SOLUTION BY ZINC

(71) Applicant: Gulbrandsen Technologies, Inc., Clinton, NJ (US)

(72) Inventors: Zijun Li, Westfield, NJ (US); Umesh J. Mistry, Gujarat (IN); Tausif Talati, Gujarat (IN); Deepak Joishar, Maharashtra (IN)

(73) Assignee: Gulbrandsen Technologies, Inc., Clinton, NJ (US)

(21) Appl. No.: 15/345,652

(22) Filed: Nov. 8, 2016

Related U.S. Application Data
(60) Provisional application No. 62/252,847, filed on Nov. 9, 2015.

(51) Int. Cl. A61K 8/28; A61K 8/27; A61Q 15/00; A61K 8/26

(52) U.S. Cl. CPC A61K 8/28; A61K 8/26; A61K 8/27; A61Q 15/00

(57) ABSTRACT

Antiperspirant active composition of enhanced efficacy containing a zinc salt and an efficacy enhancing agent selected from the group consisting of amino acid, hydroxy acid and a mixture thereof is disclosed. Specifically, a shelf-stable antiperspirant active solution containing an aluminum or aluminum-zirconium salt, a zinc salt, and the efficacy enhancing agent, which maintains the peak 4 concentration of at least 10% upon aging is disclosed. The present invention also includes methods of making the antiperspirant active solutions and formulations containing same.

### CROSS-REFERENCE TO RELATED APPLICATIONS

This application claims the benefit of the filing date of U.S. Provisional Application No. 62/252,847, filed Nov. 9, 2015, the disclosure of which is incorporated herein by reference.

### BACKGROUND OF THE INVENTION

Aluminum (Al) and aluminum-zirconium salts are used as actives for antiperspirants, and there have been various attempts to produce antiperspirant (AP) compositions with aluminum and aluminum-zirconium salts with enhanced efficacy providing greater reduction of perspiration.

The efficacy of an antiperspirant composition can be determined by the various aluminum polymers which are measured using a size exclusion chromatography, such as HPLC (high pressure liquid chromatography). HPLC is capable of resolving the aluminum into at least four distinct “peaks”, i.e., peaks 1&2, 3, 4 and 5. Sometimes, rather than peaks, the efficacy of an antiperspirant composition is described using the term “band”, i.e., bands I, II, III and IV. Generally, peak 1&2 corresponds to band I; and peaks 3, 4 and 5 correspond to bands II and III, and IV, respectively. The highest molecular weight Al polymer species are eluted first designated as peak 1&2. Peaks 3 and 4 are intermediate size Al complexes. Peak 5 is Al monomers and dimers.

Antiperspirant compositions having enhanced efficacy generally have peak 4 Al species. However, the activated antiperspirant solution having peak 4 Al species is unstable and loses efficacy in an aqueous solution, i.e. the amount of peak 4 Al species is reduced or peak 4 Al species reverts back to peak 3 Al species, upon cooling, aging or concentrating. Therefore, the currently available activated antiperspirant solution do not have a long-term shelf life, and therefore must further processed and turned into a powder form by, for example, quickly drying through freeze drying or spray drying process to preserve the enhanced efficacy. However, such additional process steps add cost and time in producing the antiperspirant active with enhanced efficacy. Such instability of the activated antiperspirant solution limits the antiperspirant active to only powder form. Therefore, there is still a need for a stable activated antiperspirant solution for the roll-on application.

Calcium and/or strontium have been added to produce antiperspirant salt solutions with improved efficacy. However, strontium is an expensive material in making the activated antiperspirant salt solution in a large scale, and calcium does not offer additional benefits such as antimicrobial and antibacterial properties. Moreover, attempts using other metals or metal salts did not have the activation effect and did not make aluminum antiperspirant salt solutions having high amount of peak 4 species or high peak 4 to peak 3 ratio (“4/3 ratio”) with enhanced efficacy with a long-term shelf-life.

The present invention aims at responding to the currently unanswered need for providing a new and improved activated aluminum antiperspirant solution having a high concentration of peaks 4, high 4/3 ratio, and/or 5 Al species, which maintains the efficacy as an aqueous solution during storage. There is also a need for an antiperspirant composition that not only has the enhanced efficacy and is shelf-stable, but also has other beneficial properties as an antiperspirant composition, such as antibacterial and antimicrobial properties.

### SUMMARY OF THE INVENTION

Described herein are activated and shelf-stable activated AP antiperspirant solution with enhanced efficacy and methods of making same.

The inventors have discovered that a basic aluminum chloride (BAC) solution also referred to as a polyaluminum solution (PAC) can be activated in the presence of a zinc salt and an efficacy enhancing agent selected from the group consisting of amino acid, hydroxy acid or a mixture thereof. The inventors have discovered that the combination of zinc and the efficacy enhancing agent selected from the group consisting of amino acid, hydroxy acid or a mixture thereof not only activates BAC, but also stabilizes the activated solution to maintain its concentration of peak 4 Al species or high peak 4/3 ratio upon aging, cooling or concentrating so that the solution stays shelf-stable with high concentration of peak 4 Al species, and maintains the efficacy as an aqueous solution. The inventors have also discovered that the concentrations of Al species and the efficacy enhancing agent have a major impact on the initial efficacy and the long-term stability and that the concentrations of Al species and the efficacy enhancing agent must be balanced to impart both the efficacy and the long-term stability of the activated solution.

In one aspect of the invention, the activated AP active solution is stable for at least 2 month, more preferably 6 month, and most preferably greater than 9 month.

In one embodiment, the preferred efficacy enhancing agent is selected from the group consisting of amino acid, hydroxy acid and a mixture thereof. In a preferred embodiment, the efficacy enhancing agent is glycine, arginine, betaine or a mixture thereof.

In one embodiment, the Al concentration of the activated AP active solution in accordance with the present invention is preferably less than 6.5%, preferably from about 0.1% to about 6%. In some embodiments, the Al concentration of the activated AP active solution is greater than 6.5%, preferably from about 0.1% to about 10%, for example, when arginine is used.

In another embodiment, the concentration of the efficacy enhancing agent of the activated AP active solution in accordance with the present invention is not more than 10%, preferably from about 1% to 8%, more preferably from 2% to 6.5%.

In one embodiment, the activated AP active solution in accordance with the present invention has a peak 4 Al concentration of at least about 10%. In another embodiment, the activated and stable antiperspirant solution in accordance with the present invention has a concentration of peak 4 of about 15% to about 50%, preferably from about 20% to about 50%.

In another embodiment, the activated AP active solution has a ratio of peak 4 Al concentration to peak 3 Al concentration (“peak 4/3”) of greater than 0.4, preferably greater than 0.5, and more preferably greater than 0.5, and most preferably greater than 0.7.

In another embodiment, the activated and stable AP active solution accordance with the present invention has both peak 4 and peak 5 Al species with low or no irritancy. Depolymerized Al species, e.g., peak 5 Al species, are believed to have enhanced efficacy. For example, AP actives containing peak 5 Al species, such as aluminum chloride, is highly effective and is used for the treatment of hyperhidrosis. The drawback for aluminum chloride, however, lies in its high irritancy. In one embodiment, the activated and stable AP active solution in accordance with the present invention has about at least about 5% of peak 5 Al species to about 80% of peak 5 Al species.

In one embodiment, the activated and stable AP active solution in accordance with the present invention does not contain calcium or strontium.

### DETAILED DESCRIPTION

The invention will be described in more detail below.

While the specification concludes with the claims particularly pointing out and distinctly claiming the invention, it is believed that the invention described herein will be better understood from the following description. All temperatures are in degrees Celsius unless specified otherwise. The invention described herein can comprise (open ended) or consist essentially of the components of the invention described herein as well as other ingredients or elements described herein. As used herein, “comprising” means the elements recited, or their equivalent in structure or function, plus any other element or elements which are not recited. The terms “having,” “including,” and “comprised of” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

It should be further understood that a description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.3, 3, 4, 5, 5.7 and 6. This applies regardless of the breadth of the range.

The term “stable” is used interchangeably with the term “shelf-stable” and “long-term shelf life” and means that the aluminum and/or aluminum-zirconium AP active solutions does not gel for at least at least 2 months, preferably 6 months and more preferably more than 1 year at room temperature, and can maintain the peak 4 Al species concentration within the range of +/−10% from the initial concentration, preferably, +/−5% for at least 2 months, preferably 6 months and more preferably more than 1 year at room temperature, and/or the peak 4/3 does not decrease to under 0.5 for at least 2 months, preferably 6 months and more preferably more than 1 year at room temperature.

The term “concentration” used with respect to peak 1&2, 3, 4 or 5 is used interchangeably with the term “amount” relative to the total concentration of peak 1&2, 3, 4, and 5. For example, a certain % of peak 4 Al species concentration is relative to the total concentration of peak 1&2, 3, 4 and 5 Al species. The concentration of peak 1&2, 3, 4 or 5 Al species is analyzed by the size exclusion chromatogram (SEC) using a high performance liquid chromatograph (HPLC) as described hereinafter. A Phoenomenex Column (3.9×300 mm, 10 μm packing) and a Waters column (μPorasil Column 3.9×300 mm, 10 μm packing) were connected in series to obtain a SEC-HPLC chromatograph. The HPLC employed is a Shimadzu RID 10A refractive index detector equipped with LC 20 AD isocratic pump and 20 μL injector. For example, to measure the concentration of a specific peak polymer in an activated aluminum solution, the solution was injected into the HPLC and eluted at a low rate of 0.9 mL/min with mobile phase of 0.01N nitric acid. For example, to measure the concentration of a specific peak polymer in activated aluminum powders, they were dissolved in DI water to form a 2% by weight Al solution and quickly injected into the HPLC and eluted at a flow rate of 0.9 mL/min with the mobile phase of 0.01N nitric acid.

The term “activated” used herein means that the aluminum and/or aluminum-zirconium AP active compositions (in a powder form or in a liquid form as the activated AP solution) has a concentration of peak 4 Al species from about 10% to about 50%, preferably from about 20% to about 40%, and/or has peak 5 Al species concentration from about 5% to about 35%, and preferably from about 10% to about 30%.

The term “solution” used herein means a liquid and does not include a gel.

The term “antibacterial” used herein means that it is capable of killing bacteria outright or a material that is able to stop additional growth of bacteria.

The term “antimicrobial” used herein means that it is capable of killing microbes outright or a material that is able to stop adding growth of microbes.

### Antiperspirant Composition

The antiperspirant (AP) composition in accordance with the present invention contains peak 4 Al species and is shelf-stable. Preferably, the AP active composition of the present invention has both high peak 4 and peak 5 Al species. The AP active composition in accordance with the present invention also contains zinc, and does not contain calcium and/or strontium. The AP active composition in accordance with the present invention further comprises an efficacy enhancing agent such as an amino acid, a hydroxy acid or a mixture thereof. Preferably, the efficacy enhancing agent is glycine, arginine, betaine or a mixture thereof. Preferably, the AP active composition in accordance with the present invention also has antibacterial and/or antimicrobial properties.

Preferably, the AP active composition in accordance with the present invention is provided in the form of a liquid, i.e., aluminum or aluminum-zirconium AP active solution. The aluminum or aluminum-zirconium AP active solution in accordance with the present invention contains zinc, and does not contain calcium or strontium. The aluminum or aluminum-zirconium AP active solution in accordance with the present invention further comprises an efficacy enhancing agent such as an amino acid, a hydroxy acid or a mixture thereof. Preferably, the efficacy enhancing agent is glycine, arginine, betaine or a mixture thereof. The aluminum or aluminum-zirconium AP active solution in accordance with the present invention is activated and shelf-stable. Preferably, the aluminum or aluminum-zirconium AP active solution in accordance with the present invention also has antibacterial properties.

The aluminum or aluminum-zirconium AP active solution in accordance with the present invention has the peak 4 concentration of at least about 10%, preferably at least about 20%, most preferably at least about 25%. In another embodiment, the aluminum or aluminum-zirconium AP active solution in accordance with the present invention has at the peak 4 concentration ranging from about 10% to about 50%, more preferably from about 20% to about 40%.

If the peak 4 concentration is less than 10%, the AP active solution is not activated and would not have the enhanced efficacy. If the peak 4 concentration is more than 50%, the AP active solution forms a gel and cannot be formulated in a topical formulation.

In another embodiment, the activated and stable antiperspirant solution in accordance with the present invention has both peak 4 and peak 5 Al species with low or no irritancy. In one embodiment, the activated and stable antiperspirant solution has the peak 5 concentration of at least about 5%, preferably at least about 10%, most preferably at least about 15%. In one embodiment, the activated and stable antiperspirant solution has the peak 5 concentration of not more than 80%. In another embodiment, the aluminum or aluminum-zirconium AP active solution in accordance with the present invention has at the peak 5 concentration ranging from about 5% to about 80%, preferably from about 15% to about 70%.

In some embodiments, the concentration of peak 4 is greater than the concentration of peak 5 in the activated AP solution. In other embodiments, the concentration of peak 5 is greater than the concentration of peak 4 in the activated AP solution.

The inventors have discovered that a basic aluminum chloride (BAC) solution also referred to as a polyaluminum solution (PAC) can be activated in the presence of a zinc salt and an efficacy enhancing agent selected from the group consisting of amino acid, hydroxy acid or a mixture thereof. The inventors have discovered that the combination of zinc and the efficacy enhancing agent selected from the group consisting of amino acid, hydroxy acid or a mixture thereof not only activates BAC, but also stabilizes the activated solution to maintain its concentration of peak 4 Al species upon aging, cooling or concentrating so that the solution stays shelf-stable with high concentration of peak 4 Al species, and maintains the efficacy as an aqueous solution. In one aspect of the invention, the activated AP active solution is stable for at least 2 month, more preferably 6 month, and most preferably greater than 9 month.

The inventors have also discovered that the concentrations of Al species and the efficacy enhancing agent have a major impact on stability. The preferred efficacy enhancing agent is glycine, In a preferred embodiment, the efficacy enhancing agent is glycine, arginine, betaine or a mixture thereof.

In one embodiment, the Al concentration of the activated AP active solution in accordance with the present invention is preferably less than 6.5%, preferably from about 0.1% to about 6%. In some embodiments, for example, when arginine is used as the efficacy enhancing agent, the Al concentration of the activated AP active solution is greater than 6.5%, preferably from about 0.1% to about 10%.

In another embodiment, the concentration of the efficacy enhancing agent of the activated AP active solution in accordance with the present invention is not more than 10%, preferably from about 1% to 8%, more preferably from 2% to 6.5%.

The antiperspirant salts of the present invention may be formulated into topical compositions such as liquids (e.g., for roll-on or porous applicators), lotions, creams, gels, soft-solids, solid sticks, aerosols, etc. Such compositions will comprise the antiperspirant salt composition in a perspiration reducing effective amount and a dermatologically acceptable carrier. The composition of the present invention can be formulated as a clear, translucent or opaque product. The preferred formulation is a clear gel or roll-on formulation made by using the aluminum or aluminum-zirconium AP active solution in accordance with the present invention.

In one embodiment, the liquid form of the aluminum or aluminum-zirconium AP active solution in accordance with the present invention may be directly utilized in oil-in water and water-in oil emulsions, and formulated as roll-on products.

In one embodiment, the AP active composition in accordance with the present invention comprises, in percent by weight, about 1% to about 80% of an aluminum or aluminum-zirconium AP salt; about 1% to about 25% of zinc; and about 1% to about 25% of an efficacy enhancing agent selected from the group consisting of amino acid, hydroxy acid and a mixture thereof.

In another embodiment, the AP active solution in accordance with the present invention comprises, in percent by weight, about 1% to about 45% of an aluminum or aluminum-zirconium AP salt; about 1% to about 20% of zinc; about 1% to about 15% of an efficacy enhancing agent selected from the group consisting of amino acid, hydroxy acid and a mixture thereof; and about 20% to about 95% of water.

In some embodiments, the AP active solution comprises about 10% to about 40% of solids, preferably about 18% to about 38% and most preferably from about 20% to about 35% relative to the total weight of the solution.

In yet another embodiment, the AP active composition in a powder form comprises about 10% to about 80%, preferably 40% to about 80%; most preferably from 50 to 70% of aluminum or aluminum-zirconium AP salt; about 2 to about 25% of zinc; and about 2 to about 25% of an efficacy enhancing agent selected from the group consisting of amino acid, hydroxy acid and a mixture thereof.

In another embodiment, the AP active solution in accordance with the present invention contains a liquid polyhydric alcohol such as propylene glycol in addition to water. In such embodiment, the antiperspirant solution comprises about 1 to about 45% of the aluminum or aluminum-zirconium AP salt; about 1% to about 20% zinc, about 1% to about 15% of an efficacy enhancing agent selected from the group consisting of amino acid, hydroxy acid and a mixture thereof, of about 10% to about 80% of water, and about 1% to about 50% of polyhydric alcohol. The AP active solution comprising polyhydric alcohol may be readily formulated as a topical antiperspirant formulation, such as a clear gel formulation.

In another embodiment, the aluminum or aluminum-zirconium AP active solution in accordance with the present invention has a viscosity ranging from about 2 cps to about 30 cps, more preferably from about 5 cps to about 10 cps.

In order to produce the AP active composition in a powder form, the aluminum or aluminum-zirconium AP active solution in accordance with the present invention is dried by, for example, via freeze drying, vacuum drying or spray drying process.

### Aluminum/Aluminum-Zirconium Salts

In one embodiment, the aluminum AP active composition in accordance with the present invention comprises the following basic aluminum salt of Formula I:

Al₂(OH)₆₋ₐXₐ, (Formula I)

wherein X is Cl, Br, I or NO₃, preferably Cl,

0<a<6, and

0≦b≦5,

In a preferred embodiment, the basic aluminum chloride has Al:Cl ratio is about 0.3 to about 2.1, preferably about 0.5 to about 1.8, and most preferably from about 0.5 to about 1.4.

Preferably, the basic aluminum salt include, without limitation, polyaluminum chloride, aluminum chlorohexahydrate, aluminum dichlorohydrate, aluminum chlorohydrate, aluminum sesquichlorohydrate, aluminum chlorohydrex PG, aluminum dichlorohydrex PG, aluminum sesquichlorohydrex PG, aluminum chlorohydrex PEG, aluminum sesquichlorohydrex PEG, aluminum chloride (15 percent or less aqueous solutions) also known as aluminum trichloride, buffered aluminum sulfate, basic aluminum chlorides or a mixture thereof.

In another embodiment, the aluminum-zirconium AP active composition in accordance with the present invention comprises a reaction product of basic aluminum salt of Formula I above and zirconium compound of the Formula II:

ZrO(OH)_{2-pc}Y_{c}, (Formula II)

wherein Y is halide, nitrate, sulfate, percholate or carbonate,

0.5≦c≦2,

p is the valence of Y; and

(2−pc)≧0.

Preferably, the zirconium salt is zirconyl hydroxychloride with the formula ZrO(OH)_{2-c}Cl_{c}, wherein c is about 0.8 to about 2, preferably about 1 to about 2. In one embodiment, the preferred aluminum-zirconium salt includes, without limitation, aluminum zirconium octachlorohydrate, aluminum-zirconium tetrachlorohydrate, aluminum-zirconium pentachlorohydrate, aluminum-zirconium trichlorohydrate or a mixture thereof. The preferred aluminum-zirconium salt is aluminum zirconium octachlorohydrate salts.

The aluminum-zirconium AP salt in accordance with the present invention has an Al:Zr ratio of about 2 to about 10, preferably of about 6 to about 10. In another embodiment, the aluminum-zirconium AP salt in accordance with the present invention has an metal:Cl ratio of about 0.7 to about 2, preferably about 0.9 to about 1.5.

In one embodiment, the Al concentration of the activated AP active solution in accordance with the present invention is preferably less than 6.5%, preferably from about 0.1% to about 6%. In some embodiments, when arginine is used as the efficacy enhancing agent, the Al concentration of the activated AP active solution is greater than 6.5%, preferably from about 0.1% to about 10%.

In one embodiment, the amount of aluminum or aluminum-zirconium salt present in the activated AP solution is from about 1 to about 45%, preferably from about 2% to about 40%, and most preferably from about 5% to about 35%.

In another embodiment, the amount of aluminum or aluminum-zirconium salt in the activated AP composition in a powder form is from about 1 to about 80%, preferably from about 40% to about 80%, and most preferably from about 50% to about 70% relative to the total weight of the composition.

### Zinc Salt

A zinc salt is used to activate the basic aluminum chloride solution in accordance with the present invention. In some embodiments, the aluminum or aluminum-zirconium AP active solution in accordance with the present invention does not contain calcium or strontium or salt thereof.

The inventors have discovered that basic aluminum chloride (BAC) solution, also known as polyaluminum chloride (PAC) solution, can also be activated in the presence of zinc salt and an efficacy enhancing agent selected from the group consisting of amino acid, hydroxy acid or a mixture thereof. The inventors have discovered that zinc in combination with the efficacy enhancing agent selected from the group consisting of amino acid, hydroxyl acid or a mixture thereof not only activates BAC, but it also stabilizes the activated solution to maintain the concentration of peak 4 Al species upon aging so that the solution stays shelf-stable with high concentration of peak 4 Al species. In one aspect of the invention, the activated AP active solution is stable for 2 month, preferably 6 months, and most preferably greater than 9 month.

In addition to activating the aluminum composition, improving the efficacy of the antiperspirant composition and stabilizing the antiperspirant solution by enabling it to maintain its enhanced efficacy as an aqueous solution, zinc salts also provide antibacterial and antimicrobial activities.

Preferred zinc salts include, without limitation, zinc oxide, zinc chloride, zinc nitrate, zinc citrate, zinc acetate, zinc lactate, zinc glycinate, zinc oxide, zinc carbonate, zinc hydroxide or a mixture thereof.

In one embodiment, the amount of zinc present in the AP active solution in accordance with the present invention is at least about 0.1% relative to the total weight of the solution. In another embodiment, the amount of zinc present in the AP active solution is from about 0.5% to about 20%, preferably from about from about 1% to about 10%, more preferably from about 1% to about 5% relative to the total weight of the solution.

In one embodiment, the amount of the zinc present in the AP active composition in a powder form is from about 1% to about 20%, preferably from about 10% to about 20% relative to the total weight of the composition.

In another embodiment, the AP active solutions in accordance with the present invention comprise an inorganic base in combination with zinc salt. Preferred inorganic bases include, without limitation, sodium hydroxide, sodium carbonate, potassium hydroxide, magnesium hydroxide and magnesium oxide.

### Efficacy Enhancing Agent

The efficacy enhancing agent in accordance with the present invention can be any material useful for increasing the amount of peak 4 species of the activated aluminum and/or aluminum-zirconium AP active solutions.

Examples of the efficacy enhancing agent, without limitation, are amino acid, hydroxy acid or a mixture thereof. The preferred amino acids are, without any limitation, glycine, arginine, alanine, valine, betaine or a mixture thereof. The preferred amino acids also include, without any limitation its corresponding compound such as alkaline glycinate, alkaline earth glycinate, zinc glycinate, urea or a mixture thereof. The preferred arginine sal includes, the arginine salt of sodium calcium, magnesium, zinc or a mixture thereof. The preferred hydroxy acids are, without any limitation, glycolic acid, lactic acid or a mixture thereof. In one embodiment, the amino acid is glycine. In another embodiment, the amino acid is glycine and arginine.

The inventors discovered that increasing the concentration of peak 4 in an aluminum or aluminum-zirconium AP active solution can be achieved by increasing the amount of the efficacy enhancing agent selected from the group consisting of amino acid, hydroxy acid and a mixture thereof.

The inventors also have discovered that having the efficacy enhancing agent allows to produce the activated and stable antiperspirant aqueous solution that has both high concentrations of peak 4 and peak 5 Al species with low or no irritancy.

The inventors have also discovered that the concentrations of Al species and the efficacy enhancing agent have a major impact on stability. In a preferred embodiment, the efficacy enhancing agent is glycine, arginine, betaine or a mixture thereof.

In another embodiment, the concentration of the efficacy enhancing agent of the activated AP active solution in accordance with the present invention is not more than 10%, preferably from about 1% to 8%, more preferably from 2% to 6.5%, and most preferably from 3% to 6% relative to total weight of the AP active solution.

In another embodiment, the amount of the efficacy enhancing agent in composition in the powder form is from about 2% to about 25%.

If the amount of the efficacy enhancing agent is less than 1%, the AP active solution is not activated and would not have the enhanced efficacy as it would not have sufficient concentrate of peak 4. If the amount of the efficacy enhancing agent is more than 10%, the AP active solution would be gelled and would be difficult to be formulated into a topical formulation.

### Method of Making

In one embodiment, the present invention provides a method for producing an activated aluminum or aluminum-zirconium AP active solution. In one embodiment, the method comprises diluting an aluminum salt-containing solution to obtain a solution containing about 20% to about 30% by weight of an aluminum salt compound relative to the total weight of the solution; heating the diluted solution; adding a zinc salt; adding an efficacy enhancing agent selected from the group consisting of amino acid, hydroxy acid and a mixture thereof. The method step order herein is irrelevant and therefore is not limited to a specific order.

In another embodiment, the method in accordance with the present invention further includes adding a zirconium compound of formula II.

In one embodiment, the preferred aluminum salt is, without limitation, aluminum trichloride, polyaluminum chloride, aluminum hexahydrate, aluminum chlorohydrate, aluminum chlorohexahydrate, aluminum dichlorohydrate, aluminum sesquichlorohydrate, aluminum chlorohydrex PG, aluminum dichlorohydrex PG, aluminum sesquichlorohydrex PG, aluminum chlorohydrex PEG, aluminum sesquichlorohydrex PEG, buffered aluminum sulfate, or a mixture thereof.

In one embodiment, the aluminum AP active solution having peak 4 species is achieved by activation of polyaluminum chloride (PAC) with Al/Cl atomic ratio of about 0.5 to about 0.6 with zinc oxide in the presence of an amino acid and/or hydroxy acid. In another embodiment, the aluminum AP active solution having peak 4 species is achieved by activation of aluminum dichlorohydrate (ADCH) with zinc oxide in the presence of an amino acid and/or hydroxy acid. Preferably, the diluted aqueous solution of PAC or ADCH is heated to about 50° C. to about 95° C. to reflux for about 1 hour to about 6 hours. The resulting Al solution has at least about 2% Al by weight, preferably at least about 4% Al by weight and most preferably at least about 5% Al by weight relative to the total weight of the aqueous solution.

### Examples

### Examples 1-4: Preparation of Aluminum AP Active Compositions in Liquid and Powder Forms with Enhanced Efficacy Containing Zinc and Glycine

Polyaluminum chloride solutions having Al/Cl ratio of about 0.55 were diluted and heated to about 90° C., different amount of glycine were added, followed by gradual addition of zinc oxide until clear solutions formed. Examples 1 and 3 were spray dried to make Examples 2 and 4, respectively, which are in powder forms. Results are listed in Table 1 below.

TABLE 1

| | % Al | % glycine | % Zn | % peak 4 | % peak 5 |
|---|---|---|---|---|---|
| Example 1 | 4.45 | 3.96 | 6.72 | 29.55 | 14.93 |
| (solution) | | | | | |
| Example 2 | 12.56 | 11.18 | 18.97 | 33.00 | 14.53 |
| (powder) | | | | | |
| Example 3 | 6.95 | 2.75 | 10.48 | 21.32 | 15.00 |
| (solution) | | | | | |
| Example 4 | 12.70 | 5.03 | 19.16 | 22.46 | 14.77 |
| (powder) | | | | | |

### Examples 5-8: Performance of Efficacy Enhancing Agent Based on Varying Amounts

Experiments were conducted to find out the effect of glycine. Similar to Examples 1-4, polyaluminum chloride solutions (PAC) solution and zinc oxide were used. We found HPLC peak 4 of the PAC solutions increased by increasing the amount of glycine. Table 2 summarizes the results.

When the AP active solution did not have any amount of the efficacy enhancing agent, i.e., glycine, the AP active solution did not have any amount of peak 4 (See Example 5). When the AP active solution had more than 10% of glycine, the AP active solution gelled and was not stable (See Example 8).

TABLE 2

| | % Al | % glycine | % Zn | % peak 4 | % peak 5 |
|---|---|---|---|---|---|
| Example 5 (not | 4.90 | 0 | 8.7 | 0 | 4.81 |
| activated) | | | | | |
| Example 6 | 4.77 | 3.05 | 8.13 | 27.84 | 4.21 |
| Example 7 | 4.60 | 6.00 | 8.50 | 31.32 | 5.00 |
| Example 8 (gelled) | 4.01 | 10.01 | 7.25 | 52.86 | 8.33 |

### Examples 9-12: Preparation of Aluminum AP Active Solutions with Enhanced Efficacy Containing Zinc, Glycine and/or Betaine

PAC-Zn-glycine and PAC-Zn-glycine-betaine solutions having both high HPLC peak 4 and peak 5 were prepared by using similar method as in Experiment 1 and the results are listed in Table 5.

TABLE 5

| | % | % | % | % | % | % |
|---|---|---|---|---|---|---|
| | Al | glycine | betaine | Zn | peak 4 | peak 5 |
| Example 9 | 7.5 | 5.0 | — | 10.5 | 28.61 | 24.74 |
| Example 10 | 7.5 | 2.5 | 2.5 | 10.5 | 23.73 | 20.63 |
| Example 11 | 7.5 | 7.0 | — | 10.5 | 33.67 | 21.88 |
| Example 12 | 7.5 | 5.0 | 2.0 | 10.5 | 30.86 | 19.45 |

Experiment 1: Stability Test

This experiment demonstrated the stability of PAC-Zn-glycine solution. Example 7 containing 4.6% AL and 6% glycine was monitored at room temperature and tested to determine whether the aqueous solution lost its initial efficacy using HPLC by measuring the concentration of peak 4 for an extended period of time. The results of the stability tests are shown below in Table 3 which demonstrated that the solution stayed stable.

TABLE 3

| Example 7 | fresh | 2 MO | 3 MO | 12 MO | 15 MO |
|---|---|---|---|---|---|
| % peak 4 | 31.32 | 28.49 | 25.69 | 25.37 | 33.11 |

Similar PAC-Zn-Betaine solution having 4% Al, 5% betaine and 6.3% Zn also demonstrated good HPLC peak 4 stability.

TABLE 4

| Example 13 | fresh | ~2 MO | 3 MO | 6 MO | ~9 MO |
|---|---|---|---|---|---|
| % peak 4 | 30.27 | 31.99 | 31.49 | 29.22 | 29.99 |

### Example 14-17: Preparation of Aluminum-Zirconium AP Active Solutions with Enhanced Efficacy Containing Zinc, Glycine and/or Betaine

Zirconium hydroxychloride solutions were added to the Al—Zn-Glycine and Al—Zn-Glycine-Betaine solutions respectively to obtain the corresponding octa Al—Zr solutions and the data are summarized in Table

TABLE 6

| | % | % | % | | % | % |
|---|---|---|---|---|---|---|
| | Al | Zr | Zn | Al/Zr | peak 4 | peak 5 |
| Example 14 | 6.32 | 2.52 | 9.21 | 8.64 | 22.63 | 25.84 |
| (glycine only) | | | | | | |
| Example 15 | 6.21 | 2.48 | 9.15 | 8.63 | 20.52 | 23.16 |
| (glycine & betaine) | | | | | | |
| Example 16 | 6.42 | 2.40 | 8.92 | 9.22 | 30.29 | 22.82 |
| (glycine only) | | | | | | |
| Example 17 | 6.58 | 2.44 | 8.80 | 9.29 | 28.39 | 20.56 |
| (glycine & betaine) | | | | | | |

Experiment 2: Stability Test Based on Concentrations of Al and Glycine

This experiment demonstrated that Al concentration and glycine concentration play a key role in the stability of Al—Zn-glycine and Al—Zr—Zn-glycine solutions. The solutions containing % Al of no more than 6.5% and % glycine of no more than 6.5% stayed stable for more than 200 days as demonstrated in the below examples.

| | Al/Cl Ratio | | | | Stability |
|---|---|---|---|---|---|
| | (atomic) | % Al | % Zn | % glycine | <10 cp |
| Example 18 | 0.7 | 6.21 | 6.94 | 3.11 | 285 days |
| Example 19 | 0.8 | 6.02 | 6.20 | 3.01 | >365 days |
| Example 20 | 1.2 | 6.40 | 2.37 | 6.40 | 330 days |
| Example 21 | 1.4 | 6.25 | 1.52 | 6.25 | >360 days |

The glycine concentration can stay as low as 2% and the solution stayed stable as shown below:

| | % Al | % Zn | % glycine | Stability |
|---|---|---|---|---|
| Example 22 | 6.5 | 1.50 | 2.00 | 5 cp, >200 days |

### Comparative Example

We have found the basic aluminum chloride solutions, when activated by zinc salts in the presence of glycine, gelled quickly at high Al concentration of greater than 6.5%. The corresponding Al—Zr solutions also gelled in a short period of time, in 15 days, as demonstrated in tables 7 & 8.

TABLE 7

| | % Al | % Zn | % Glycine |
|---|---|---|---|
| Comparative | 8.00 | 1.85 | 6.15 |
| Example 1 | | | |

| No. of Days of aging | Viscosity | % peak 5 | Peak 4/3 |
|---|---|---|---|
| 0 | 7 cps | 65.8 | 0.45 |
| 15 | Gel | — | — |

TABLE 8

| | | | | Al/Zr |
|---|---|---|---|---|
| | % Al | % Zn | % Glycine | Ratio |
| Comparative | 6.49 | 1.49 | 5.00 | 9.09 |
| Example 2 | | | | |

| No. of Days of aging | Viscosity | % peak 5 | Peak 4/3 |
|---|---|---|---|
| 0 | 5 cps | 65.5 | 0.47 |
| 30 | 6 cps | 74.3 | 0.57 |
| 60 | 589 cps | 72.9 | 0.58 |
| 75 | Gel | — | — |

By introduction of another amino acid, i.e. arginine, the stability of basic aluminum chloride solution with zinc salt at higher concentration increased substantially, especially for the corresponding Al—Zr solution shown in table 9 & 10.

TABLE 9

| | % Al | % Zn | % Arginine |
|---|---|---|---|
| Example 23 | 8.05 | 1.86 | 6.17 |

| No. of Days of aging | Viscosity | % peak 5 | Peak 4/3 |
|---|---|---|---|
| 0 | 5 cps | 59.3 | 0.41 |
| 30 | 5 cps | 65.8 | 1.15 |
| 60 | 5 cps | 67.6 | 1.35 |
| 120 | 5 cps | 67.1 | 1.43 |
| 300 | 6 cps | — | — |

TABLE 10

| | | | | A/Zr |
|---|---|---|---|---|
| | % Al | % Zn | % Arginine | Ratio |
| Example 24 | 6.51 | 1.51 | 5.00 | 9.23 |

| No. of Days of aging | Viscosity | % peak 5 | Peak 4/3 |
|---|---|---|---|
| 0 | 5 cps | 58.1 | 0.49 |
| 30 | 5 cps | 68.6 | 0.59 |
| 60 | 5 cps | 71.0 | 0.72 |
| 120 | 5 cps | — | — |
| 300 | 6 cps | — | — |

We also found the Al—Zn and Al—Zr—Zn solutions can be stabilized by mixed amino acids such as arginine and glycine. Data are shown in Tables 11&12.

TABLE 11

| | % Al | % Zn | % Arginine | % Glycine |
|---|---|---|---|---|
| Example 25 | 7.95 | 1.99 | 2.00 | 2.00 |

| No. of Days of | | | |
|---|---|---|---|
| aging | Viscosity | % peak 5 | Peak 4/3 |
| 0 | 5 cps | 58.3 | 0.24 |
| 30 | 5 cps | 64.6 | 0.70 |
| 90 | 5 cps | 67.5 | 0.88 |
| 180 | 8 cps | 64.3 | 0.73 |
| 240 | 17 cps | — | — |

We also found the Al—Zn and Al—Zr—Zn solutions in the presence of both arginine and glycine have better stability in comparison to glycine alone as shown in Tables 12&13.

TABLE 12

| | % Al | % Zn | % Arginine | % Glycine | A/Zr |
|---|---|---|---|---|---|
| Example 26 | 6.49 | 1.65 | 1.65 | 1.65 | 9.00 |

| No. of Days | | | |
|---|---|---|---|
| of aging | Viscosity | % peak 5 | Peak 4/3 |
| 0 | 5 cps | 59.9 | 0.26 |
| 30 | 5 cps | 74.2 | 0.76 |
| 90 | 5 cps | — | — |
| 180 | 8 cps | 78.2 | 1.13 |
| 240 | 15 cps | — | — |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An antiperspirant active solution comprising:
an aluminum or aluminum-zirconium salt,
a zinc salt, and
an efficacy enhance agent selected from the group consisting of amino acid, hydroxy acid and a mixture thereof;
wherein a concentration of peak 4 is about 10% to about 50% and the concentration of peak 5 is about 5 to about 80%, wherein the solution is shelf-stable.

2. The antiperspirant active solution of claim 1, wherein the solution is antibacterial and/or antimicrobial.

3. The antiperspirant active solution of claim 1, wherein the solution does not comprise calcium or strontium.

4. The antiperspirant active solution of claim 1, wherein the concentration of peak 4 is from about 20% to about 40%.

5. The antiperspirant active solution of claim 1, wherein the concentration of peak 5 is from about 10% to about 70%.

6. The antiperspirant active solution of claim 1, wherein a ratio of peak 4 Al concentration to peak 3 Al concentration (“peak 4/3”) is greater than 0.4.

7. The antiperspirant active solution of claim 1, wherein the aluminum or aluminum-zirconium salt is present in an amount of about 0.1% to about 20%; the zinc is present in an amount of about 1% to about 20%, the efficacy enhancing agent is present in an amount of about 1% to about 10%; and the water is present in an amount of about 5% to about 95% of the water.

8. The antiperspirant active solution of claim 1, wherein the solution further comprises polyhydric alcohol.

9. The antiperspirant active solution of claim 1, wherein the aluminum salt is polyaluminum chloride, aluminum trichloride, aluminum chlorohexahydrate, aluminum dichlorohydrate, aluminum chlorohydrate, aluminum sesquichlorohydrate, aluminum chlorohydrex PG, aluminum dichlorohydrex PG, aluminum sesquichlorohydrex PG, aluminum chlorohydrex PEG, aluminum sesquichlorohydrex PEG, buffered aluminum sulfate, basic aluminum chlorides or a mixture thereof.

10. The antiperspirant active solution of claim 1, wherein the aluminum-zirconium salt is aluminum zirconium octachlorohydrate.

11. The antiperspirant active solution of claim 1, wherein the zinc salt is zinc oxide, zinc chloride, zinc nitrate, zinc citrate, zinc acetate, zinc lactate, zinc glycinate, zinc oxide, zinc carbonate, zinc hydroxide or a mixture thereof.

12. The antiperspirant active solution of claim 1, wherein the amino acid is glycine, arginine, alanine, valine, betaine or a mixture thereof.

13. The antiperspirant active solution of claim 1, wherein the hydroxy acid is glycolic acid, lactic acid or a mixture thereof.

14. A topical composition comprising an effective amount of the antiperspirant active solution of claim 1 and a dermatologically acceptable carrier.

15. The topical composition of claim 14 in the form of a liquid for roll-on or porous applicator, lotion, cream, gel, soft-solid, solid stick or aerosol.

16. A method of reducing perspiration from human skin and providing antibacterial activities comprising applying to human skin a topical composition of claim 14.

17. A method of preparing an efficacy enhanced and shelf-stable antiperspirant active solution comprising:
diluting an aluminum salt-containing solution to obtain a diluted solution containing about 20% to about 30% by weight of an aluminum salt compound relative to the total weight of the solution;
heating the diluted solution;
adding a zinc salt;
adding an efficacy enhancing agent selected from the group consisting of amino acid, hydroxy acid and a mixture thereof; and
optionally adding a zirconium compound.

18. The method of claim 17, wherein the concentration of peak 4 of the activated solution is about 10% to about 50% and the concentration of peak 5 is about 2 to about 80%.

19. The method of claim 17, wherein the method does not include adding calcium or strontium.

20. The method of claim 17, wherein a ratio of peak 4 Al concentration to peak 3 Al concentration (“peak 4/3”) is greater than 0.4.
